(19) United States
(12) Patent Application Publication
(10) Pub. No.: US 2017/0105681 A1
(43) Pub. Date: Apr. 20, 2017

(54) METHOD AND DEVICE FOR NON-INVASIVE MONITORING OF PHYSIOLOGICAL PARAMETERS

(71) Applicant: XEROX CORPORATION, Norwalk, CT (US)

(72) Inventors: Mridula Singh, Uttar Pradesh (IN); Kuldeep Yadav, Gurgaon (IN); Abhishek Kumar, New Delhi (IN); HIMANSHU J. MADHU, Borivali (West) (IN); Tridib Mukherjee, Bangalore (IN)

(21) Appl. No.: 14/886,165

(22) Filed: Oct. 19, 2015

(51) Int. Cl. A61B 5/00; A61B 5/16; A61B 5/103; A61B 5/0205

(52) U.S. Cl. CPC A61B 5/6897; A61B 5/02055; A61B 5/486; A61B 5/4884; A61B 5/165; A61B 5/0077; A61B 5/441; A61B 5/1032; A61B 5/7264; A61B 5/7282; A61B 2562/0204; A61B 2562/0271; A61B 2090/378

(57) ABSTRACT

A method and a system are provided for monitoring a physiological parameter of a user. The method comprises monitoring an operation performed on one or more input devices associated with an electronic device and one or more applications being accessed on the electronic device using one or more first sensors for a pre-defined time frame. The method utilizes one or more processors to identify a current time instant based on the monitoring of the operation. The one or more processors further determine whether the current time instant may correspond to an opportune time instant by utilizing a classifier. In an embodiment, the physiological parameter of the user may be monitored at the current time instant when the current time instant corresponds to the opportune time instant.

### TECHNICAL FIELD

The presently disclosed embodiments are related, in general, to diagnostic systems. More particularly, the presently disclosed embodiments are related to a method and a device for non-invasive monitoring of one or more physiological parameters.

### BACKGROUND

Diagnostic systems may enable monitoring of one or more physiological parameters of a human subject. Usually diagnostic systems may utilize invasive techniques to monitor the one or more physiological parameters of the human subject. Advancements in the field of diagnosis and image processing have enabled monitoring of the one or more physiological parameters using non-invasive techniques. For example, a digital camera may be utilized to monitor one or more features, such as skin tone, associated with the human subject. Based on the subtle color changes in the skin tone of the human subject cardiac conditions of the human subject may be determined.

To monitor the one or more features associated with the human subject through the digital camera, appropriate lighting conditions may be required. Further, it may be necessary that the human subject remains stable during which the human subject is monitored. As the human subject has the knowledge that he/she is being monitored through the digital camera, the human subject may feel intrusive. This may further lead to inaccurate determination of the one or more physiological parameters based on the monitored one or more features.

Further limitations and disadvantages of conventional and traditional approaches will become apparent to those skilled in the art, through a comparison of described systems with some aspects of the present disclosure, as set forth in the remainder of the present application and with reference to the drawings.

### SUMMARY

According to embodiments illustrated herein, there may be provided a method for monitoring a physiological parameter of a user. The method may comprise monitoring an operation performed on one or more input devices associated with an electronic device and one or more applications being accessed on the electronic device using one or more first sensors for a pre-defined time frame. The method may utilize one or more processors to identify a current time instant based on the monitoring of the operation and the one or more applications being accessed on the electronic device. The one or more processors may further determining whether the current time instant may correspond to an opportune time instant by utilizing a classifier. In an embodiment, the physiological parameter of the user may be monitored at the current time instant when the current time instant corresponds to the opportune time instant.

According to embodiments illustrated herein, there may be provided an electronic device that comprises one or more processors configured to monitor a physiological parameter of a user. The one or more processors may be configured to monitor an operation performed on one or more input devices associated with the electronic device and one or more applications being accessed on the electronic device for a pre-defined time frame. The one or more processors may be configured to identify a current time instant based on the monitoring of the operation and the one or more applications being accessed on the electronic device. The one or more processors may be further configured to determine whether the current time instant corresponds to an opportune time instant by utilizing a classifier. In an embodiment, the physiological parameter of the user may be monitored at the current time instant when the current time instant corresponds to the opportune time instant.

According to embodiments illustrated herein, a non-transitory computer-readable storage medium having stored thereon, a set of computer-executable instructions for causing a computer comprising one or more processors to perform steps of monitoring an operation performed on one or more input devices associated with an electronic device and one or more applications being accessed on the electronic device using one or more first sensors for a pre-defined time frame. The one or more processors may further identify a current time instant based on the monitoring of the operation and the one or more applications being accessed on the electronic device. The one or more processors may further determine whether the current time instant corresponds to an opportune time instant by utilizing a classifier. In an embodiment, the physiological parameter of the user may be monitored at the current time instant when the current time instant corresponds to the opportune time instant.

### BRIEF DESCRIPTION OF DRAWINGS

The accompanying drawings illustrate the various embodiments of systems, methods, and other aspects of the disclosure. Any person with ordinary skill in the art will appreciate that the illustrated element boundaries (e.g., boxes, groups of boxes, or other shapes) in the figures represent one example of the boundaries. In some examples, one element may be designed as multiple elements, or multiple elements may be designed as one element. In some examples, an element shown as an internal component of one element may be implemented as an external component in another, and vice versa. Further, the elements may not be drawn to scale.

Various embodiments will hereinafter be described in accordance with the appended drawings, which are provided to illustrate and not limit the scope in any manner, wherein similar designations denote similar elements, and in which:

FIG. 1 is a block diagram that illustrates a system environment in which various embodiments of the disclosure may be implemented;

FIG. 2 is a block diagram that illustrates an electronic device, in accordance with at least one embodiment;

FIG. 3 illustrates a block diagram to train a classifier, in accordance with at least one embodiment;

FIG. 4 is a block diagram that illustrates an exemplary scenario to monitor heart rate of the user, in accordance with at least one embodiment;

FIG. 5 is a flowchart that illustrates a method to train a classifier, in accordance with at least one embodiment; and

FIG. 6 is a flowchart that illustrates a method to monitor one or more physiological parameters, in accordance with at least one embodiment.

### DETAILED DESCRIPTION

The present disclosure may be best understood with reference to the detailed figures and description set forth herein. Various embodiments are discussed below with reference to the figures. However, those skilled in the art will readily appreciate that the detailed descriptions given herein with respect to the figures are simply for explanatory purposes, as the methods and systems may extend beyond the described embodiments. For example, the teachings presented and the needs of a particular application may yield multiple alternative and suitable approaches to implement the functionality of any detail described herein. Therefore, any approach may extend beyond the particular implementation choices in the following embodiments described and shown.

References to “one embodiment,” “at least one embodiment,” “an embodiment,” “one example,” “an example,” “for example,” and so on indicate that the embodiment(s) or example(s) may include a particular feature, structure, characteristic, property, element, or limitation but that not every embodiment or example necessarily includes that particular feature, structure, characteristic, property, element, or limitation. Further, repeated use of the phrase “in an embodiment” does not necessarily refer to the same embodiment.

### DEFINITIONS

The following terms shall have, for the purposes of this application, the respective meanings set forth below.

A “physiological parameter” refers to clinical features associated with a human subject. In an embodiment, the one or more physiological parameters associated with the human subject may have an associated data type. Examples of the data type may include, but are not limited to, a binary data type (e.g., gender, parameters related to past addictions, past diseases, and past medications), a categorical data type (e.g., education level, job type, and parameters related to radiological results), and a numerical data type (e.g., age, and parameters related to blood investigation results). Examples of a physiological parameter may include, but are not limited to, a cholesterol level, a heart rate, a blood pressure, a breath carbon-dioxide concentration, a breath oxygen concentration, a stroke score, a blood creatinine level, a blood albumin level, a blood sodium level, a total blood count, a blood glucose/sugar level, a blood hemoglobin level, and a blood platelet count.

“One or more first sensors” refers to a device that detects/measures/monitors an activity associated with one or more input devices such as a keyboard, a mouse, a touch pad, a track ball, associated with an electronic device. Further, the one or more first sensors monitor a presence of the user, a motion of the user, and an ambience around the electronic device. Examples of the one or more first sensors comprise a pressure sensor, a motion sensor, and/or the like.

“One or more second sensors” refers to a device that detects/measures/monitors events or changes in quantities and provides a corresponding output, generally as an electrical or an optical signal. In medical science, the one or more second sensors are configured to detect biological, physical, and/or chemical signals associated with a user and measure and record such signals. For example, an image sensor, a temperature sensor, a light sensor, an audio sensor and/or an ultrasonic sensor are used to monitor a physiological parameter of a user.

“One or more input devices” refers to a device that is utilized to interact with an electronic device. In an embodiment, the interaction correspond to one or more events performed on the one or more input devices. In an embodiment, the one or more the events correspond to a click operation, a scroll operation, a touch operation, and the like. Examples of the one or more input devices comprise a keyboard, a mouse, a track pad, a track ball, and/or a touch display.

A “usage pattern” refers to a log of one or more operations performed on an electronic device within a pre-defined time frame. In an embodiment, the log may comprise a count of the one or more operations performed on the one or more input devices associated with the electronic device. Further, the log may comprise at least information pertaining to an application being accessed by the user. In an embodiment, the one or more operations may corresponds to a click operation, a scroll operation, a touch operation, and the like. Examples of the usage pattern may comprise a number of left clicks performed using a mouse, a number of keys pressed on the keyboard, and an application name (e.g., a name of the web browser).

A “first time instant” refers to a time stamp associated with the monitoring of the operation being performed by the user on the one or more input devices associated with the electronic device. Further, the first time instant may correspond to the time instant at which one or more applications being accessed by the user are monitored. In an embodiment, the data captured at the first time instant, based on the monitoring of the operation and the one or more applications being accessed, is used for training a classifier. In an embodiment, training data is generated based on the first time instances.

An “opportune time instant” refers to a time stamp at which monitoring of a user is initiated using one or more second sensors. The user is monitored for a pre-defined time duration at the opportune time instant to obtain a set of values. In an embodiment, a measure of a physiological parameter is determined based on the set of values.

A “measure of a physiological parameter” refers to a value associated with a physiological parameter. Examples of a measure of a physiological parameter may include a heart rate, respiratory rate, emotions, stress, and/or the like.

A “feedback” refers to a Boolean value that is indicative of whether a measure of a physiological parameter is determined successfully at the opportune time instant. In an embodiment, the feedback is utilized to train a classifier in such a manner that the opportune time instant is determined accurately.

A “classifier” refers to a mathematical model that may be configured to categorize data into one or more categories. In an embodiment, the classifier is trained based on historical/training data. Examples of the classifier may include, but are not limited to, a Support Vector Machine (SVM), a Logistic Regression, a Bayesian Classifier, a Decision Tree Classifier, a Copula-based Classifier, a K-Nearest Neighbors (KNN) Classifier, or a Random Forest (RF) Classifier.

“Training” refers to a process of updating/tuning a classifier using feedback such that the classifier is able to determine an opportune time instant accurately.

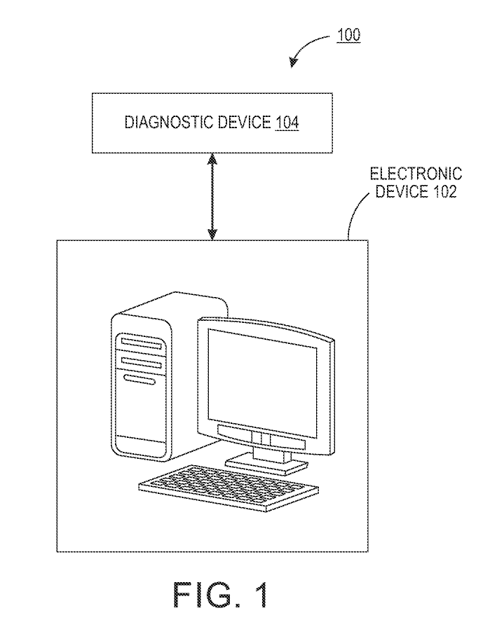

FIG. 1 is a block diagram that illustrates a system environment 100 in which various embodiments of the disclosure may be implemented. The system environment 100 may include an electronic device 102 and a diagnostic device 104. The electronic device 102 and the diagnostic device 104 may be communicatively connected to each other via a wired or wireless connection. The electronic device 102 and the diagnostic device 104 may communicate with each other via a data bus of the electronic device 102.

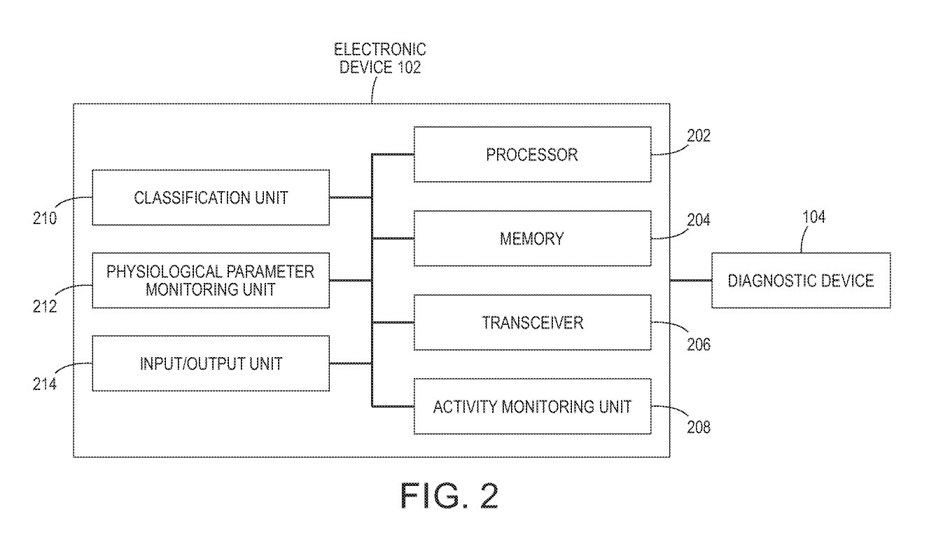

In an embodiment, the electronic device 102 may refer to a computing device used by a user. The electronic device 102 may comprise one or more processors and one or more memories. The one or more memories may include a computer readable code that may be executable by the one or more processors to perform predetermined operations. In an embodiment, the electronic device 102 may be configured to monitor an operation performed on one or more input devices associated with the electronic device 102 and one or more applications being accessed on the electronic device 102 at a current time instant. The electronic device 102 may determine whether a physiological parameter of the user can be determined at the current time instant. In an embodiment, the electronic device 102 may utilize a classifier to determine whether the physiological parameter of the user can be measured at the current time instant. In an embodiment, prior to the determination based on the classifier, the electronic device 102 may train the classifier using the training data. In an embodiment, the training of the classifier has been described later in conjunction with FIG. 2. In an embodiment, the electronic device 102 may present a user-interface to a user to display the measure of the physiological parameter of the user based on the monitoring of the user at the current time instant. In an embodiment, the electronic device 102 may include hardware and/or software to display the measure of the physiological parameter of the user. Examples of the electronic device 102 may include, but are not limited to, a personal computer, a laptop, a personal digital assistant (PDA), a mobile device, a tablet, or any other computing device.

In an embodiment, the diagnostic device 104 is a device that comprises one or more second sensors. In an embodiment, the operation of the diagnostic device 104 is controlled based on the instructions received from the electronic device 102. For example, the diagnostic device 104 may receive the instruction to initiate monitoring of the user at the current time instant. In an embodiment, the monitoring of the user may comprise capturing of a video of the user at the current time instant. Further, the diagnostic device 104 may be configured to capture supplementary information associated with the user using the one or more second sensors. Examples of the one or more second sensors comprise an image sensor, a temperature sensor, a light sensor, an audio sensor, and/or an ultrasonic sensor. In an embodiment, the captured video and the supplementary information may be stored in a storage medium, such as a film, and/or a storage device. In an embodiment, a removable storage device, such as an integrated circuit memory card may be utilized to store the video. In an embodiment, the diagnostic device 104 may be configured to implement one or more facial recognition techniques to detect the user being monitored. Examples of the diagnostic device 104 may comprise a digital camera, a surveillance camera, a webcam, and a video camera.

A person skilled in the art would understand that the scope of the disclosure should not be limited to the electronic device 102 or the diagnostic device 104 as a separate entity. In an embodiment, the functionalities of the electronic device 102 or the diagnostic device 104 may be combined into a single device without limiting the scope of the inventions.

FIG. 2 is a block diagram that illustrates the electronic device 102, in accordance with at least one embodiment. FIG. 2 is explained in conjunction with the elements from FIG. 1.

In an embodiment, the electronic device 102 includes a processor 202, a memory 204, a transceiver 206, an activity monitoring unit 208, a classification unit 210, a physiological parameter monitoring unit 212, and an input/output unit 214. The processor 202 may be communicatively connected to the memory 204, the transceiver 206, the activity monitoring unit 208, the classification unit 210, the physiological parameter monitoring unit 212, and the input/output unit 214. The transceiver 206 may be communicatively connected to the diagnostic device 104.

The processor 202 comprises suitable logic, circuitry, interfaces, and/or code that may be configured to execute a set of instructions stored in the memory 204. The processor 202 may be implemented based on a number of processor technologies known in the art. The processor 202 may work in coordination with the transceiver 206, the activity monitoring unit 208, the classification unit 210, the physiological parameter monitoring unit 212, and the input/output unit 214, to monitor the user to determine the measure of the physiological parameter. Examples of the processor 202 include, but not limited to, an X86-based processor, a Reduced Instruction Set Computing (RISC) processor, an Application-Specific Integrated Circuit (ASIC) processor, a Complex Instruction Set Computing (CISC) processor, and/or other processors.

The memory 204 comprises suitable logic, circuitry, interfaces, and/or code that may be configured to store the set of instructions, which are executed by the processor 202. In an embodiment, the memory 204 may be configured to store one or more programs, routines, or scripts that may be executed in coordination with the processor 202. In an embodiment, one or more techniques to determine the measure of the physiological parameter may be stored in the memory 204. The memory 204 may be implemented based on a Random Access Memory (RAM), a Read-Only Memory (ROM), a Hard Disk Drive (HDD), a storage server, and/or a Secure Digital (SD) card.

The transceiver 206 comprises suitable logic, circuitry, interfaces, and/or code that may be configured to receive the captured video and the supplementary information from the diagnostic device 104. The transceiver 206 may implement one or more known technologies to support wired or wireless communication with the diagnostic device 104. In an embodiment, the transceiver 206 may correspond, but is not limited to, an antenna, a radio frequency (RF) transceiver, one or more amplifiers, a tuner, one or more oscillators, a digital signal processor, a Universal Serial Bus (USB) device, a coder-decoder (CODEC) chipset, a subscriber identity module (SIM) card, and/or a local buffer. The transceiver 206 may communicate via wireless communication with networks, such as the Internet, an Intranet and/or a wireless network, such as a cellular telephone network, a wireless local area network (LAN) and/or a metropolitan area network (MAN). The wireless communication may use any of a plurality of communication standards, protocols and technologies, such as: Global System for Mobile Communications (GSM), Enhanced Data GSM Environment (EDGE), wideband code division multiple access (W-CDMA), code division multiple access (CDMA), time division multiple access (TDMA), 2G, 3G, 4G, Bluetooth, Wireless Fidelity (Wi-Fi) (e.g., IEEE 802.11a, IEEE 802.11b, IEEE 802.11g and/or IEEE 802.11n), voice over Internet Protocol (VoIP), Wi-MAX, a protocol for email, instant messaging, and/or Short Message Service (SMS).

The activity monitoring unit 208 comprises suitable logic, circuitry, interfaces, and/or code that may be configured to monitor the operation performed on the one or more input devices associated with the electronic device 102. In an embodiment, the activity monitoring unit 208 may comprise one or more first sensors that may enable the monitoring of the operation performed on the one or more input devices. In an embodiment, the operation that may be monitored by the one or more first sensors may comprise a click event, a scroll event, a typing event, and a touch event. Further, the activity monitoring unit 208 may monitor the one or more applications being accessed on the electronic device 102. In an embodiment, the activity monitoring unit 208 may generate a usage pattern of the operation performed on the one or more input devices and the one or more applications accessed by the user. In an embodiment, the activity monitoring unit 208 may further store the timestamp associated with the operations performed on the one or more input devices and the one or more applications accessed by the user in the usage pattern. In an embodiment, the timestamp recorded by the activity monitoring unit 208 may correspond to the current time instant. In an embodiment, the activity monitoring unit 208 may store the usage pattern as a JavaScript Object Notation (JSON). In an embodiment, the monitoring of the input devices may be implemented using one or more programming languages, such as C, C++, C#, Java, and the like. The activity monitoring unit 208 may be implemented as an Application-Specific Integrated Circuit (ASIC) microchip designed for a special application, such as to generate the usage pattern of the electronic device 102 based on the monitoring.

The classification unit 210 comprises suitable logic, circuitry, interfaces, and/or code that may be configured to determine whether the physiological parameter of the user can be measured at the current time instant based on a rule set determined by the classifier. Hereinafter, the current time instant at which the physiological parameter of the user can be measured has been referred to as an opportune time instant. In an embodiment, the classification unit 210 comprises the classifier. In an embodiment, the classification unit 210 may be implemented as an Application-Specific Integrated Circuit (ASIC) microchip designed for a special application, such as to determine whether the current time instant corresponds to the opportune time instant.

The physiological parameter monitoring unit 212 comprises suitable logic, circuitry, interfaces, and/or code that may be configured to determine a set of values based on analysis of the video and the supplementary information obtained from the diagnostic device 104 at the opportune time instant. Further, the physiological parameter monitoring unit 212 may be configured to determine the measure of the physiological parameter based on the set of values. In an embodiment, the physiological parameter monitoring unit 212 may utilize of one or more known techniques, such as a heart rate determination technique to determine the measure of the physiological parameter. In an embodiment, the physiological parameter monitoring unit 212 may be implemented as an Application-Specific Integrated Circuit (ASIC) microchip designed for a special application, such as to determine the measure of the physiological parameter. In an embodiment, the physiological parameter monitoring unit 212 may analyze the video and the supplementary information by using one or more programming languages such as, C, C++, C#, Java, and the like.

The input/output unit 214 comprises suitable logic, circuitry, interfaces, and/or code that may be configured to receive an input or provide an output to the electronic device 102. In an embodiment, the input/output unit 214 may display the measure of the physiological parameter on a display screen of the electronic device 102. The input/output unit 214 comprises various input and output devices that are configured to communicate with the processor 202. Examples of the input devices include, but are not limited to, a keyboard, a mouse, a joystick, a touch screen, a microphone, a camera, and/or a docking station. Examples of the output devices include, but are not limited to, a display screen and/or a speaker.

In operation, the processor 202 works in coordination with the memory 204, the transceiver 206, the activity monitoring unit 208, the classification unit 210, the physiological parameter monitoring unit 212, and the input/output unit 214 to monitor the physiological parameter of the user.

In an embodiment, the activity monitoring unit 208 may be configured to monitor one or more operations performed on the one or more input devices, by the user, using the one or more first sensors for a pre-defined time frame. In an embodiment, the one or more operations may comprise the click event, the scroll event, the typing event, and the touch event. In an embodiment, the one or more first sensors may monitor a keyboard activity of the user, a mouse activity of the user, a touch pad activity of the user, a track ball activity of the user, a presence of the user, a motion of the user, and/or an ambience, to determine the one or more operations. Examples of the one or more first sensors may comprise a pressure sensor, and a motion sensor.

For instance, the pressure sensor may be utilized to monitor the keyboard activity and/or mouse activity. In an embodiment, the activity monitoring unit 208 may determine whether the user has been active on the keyboard for the pre-defined time frame based on an input received from the pressure sensor. Further, the activity monitoring unit 208 may determine a number of keys pressed on the keyboard during the pre-defined time frame. For example, the activity monitoring unit 208 may determine that the user had pressed 20 keys in the time frame of 10 seconds. In an embodiment, the activity monitoring unit 208 may determine a number of left clicks and/or right clicks performed on the mouse within the pre-defined time frame. For example, the activity monitoring unit 208 may determine that the user had performed 10 right clicks and 20 left clicks within the time frame of 10 seconds.

Further, the activity monitoring unit 208 may be configured to monitor the one or more applications being accessed on the electronic device 102 within the pre-defined time frame. The activity monitoring unit 208 may extract information pertaining to the application being accessed on the electronic device 102 by the user. The information may comprise, but not limited to, an application title, and a time stamp at which the application is accessed. In an embodiment, the activity monitoring unit 208 may monitor the keyboard activity and/or mouse activity and the one or more applications being accessed on the electronic device 102 in real-time, for the predefined time frame.

Based on the extracted information, the activity monitoring unit 208 may be configured to categorize the one or more applications being accessed in one or more application categories. Examples of the one or more application categories may comprise, but not limited to, a browser, a file explorer, a coding IDE, email. For example, the application being accessed by the user within the pre-defined time frame is Internet Explorer. Thus, the activity monitoring unit 208 may determine that the application category of Internet Explorer is browser. The activity monitoring unit 208 may be further configured to extract detailed information associated with the application being accessed based on the extracted information. For example, the detailed information may contain information that the user is watching a video with title “XYZ” on a YouTube website by using the browser Internet Explorer.

Based on the monitoring of the operations performed by each of the one or more input devices, and the one or more applications being accessed by the user, the activity monitoring unit 208 may be configured to determine the usage pattern of the user. In an embodiment, the activity monitoring unit 208 may store the usage pattern as a JSON object. An example of the JSON object generated based on the monitoring of the operations may be illustrated as follows:

{
“user”: “XRXEU\\q409MNMQ,
“active_on_mouse”: 1,
“no_of_mouse_clicks”: 7,
“active_on_keyboard”: 0,
“no_of_key_events”: 0,
“open_application_title”: “Inbox (1) - abc@xyz.com - email1 -
internet explorer”,
“timestamp”: “2015-02-20 12:28:33”
}

The example JSON object comprises information of the operation performed on the mouse. In an embodiment, the information may comprise that a number of mouse clicks is 7. Further, the example JSON object illustrates that no operation has been performed on the keyboard and a number of key press events is zero. Further, the application that is being accessed is Internet Explorer and the user is accessing the inbox of “email1” account with email id “abc@xyz.com”. Additionally, the JSON object captures the timestamp “2015-02-20 12:28:33” which denotes the time at which the application is accessed. In an embodiment, the captured timestamp in the JSON object corresponds to the current time instant.

A person having ordinary skills in the art will appreciate that the scope of the disclosure is not limited to store the usage pattern as JSON object. In an embodiment, the usage pattern may be stored in any other format such as, but are not limited to, XML, JScript, and/or the like.

In an embodiment, the classification unit 210 may determine whether the current time instant corresponds to the opportune time instant using a rule set defined by the classifier. Prior to determination of whether the current time instant corresponds to the opportune time instant, the processor 202 is configured to train the classifier based on the training data. In an embodiment, the processor 202 may be further configured to generate the training data.

For generating the training data, the processor 202 may instruct the activity monitoring unit 208 to monitor the operations (performed on the one or more input devices) and the one or more applications being accessed, continuously. As discussed supra, the activity monitoring unit 208 may monitor the operations and the one or more applications during one or more time frames. For each time frame, the activity monitoring unit 208 may generate the usage pattern. Further, based on the usage pattern, the activity monitoring unit 208 may determine one or more first time instants by extracting the timestamp from the usage pattern determined for each of the one or more time frames.

Concurrently, the processor 202 may instruct the diagnostic device 104 to capture the video and the supplementary information of the user at the one or more first time instants. Based on the video and the supplementary information, the physiological parameter monitoring unit 212 may be configured to determine the set of values associated with one or more features of the user. In an embodiment, the one or more features may comprise, but are not limited to, a skin tone, facial features, a measure of temperature, and/or the like. Based on the set of values, the physiological parameter monitoring unit 212 may be configured to determine a measure of the physiological parameter. Further, the measure of the physiological parameter may be compared to a pre-defined threshold associated with the physiological parameter. The physiological parameter monitoring unit 212 may determine the accuracy of the measure of the physiological parameter based on the comparison. For example, the physiological parameter corresponds to a blood pressure of the user. A normal range of the blood pressure for a human being is 120/80. For instance, based on the set of values, the physiological parameter monitoring unit 212 may determine the blood pressure as 40/20, which may not correspond to an accurate measurement of the blood pressure. In an embodiment, the predefined threshold value may be determined by an expert in the field of subject.

After determination of the accuracy of the measurement of the physiological parameter, in an embodiment, a Boolean value may be assigned to each of the one or more first time instants that may be indicative of whether the measure of the physiological parameter was determined accurately at each of the one or more first time instants. For example, the Boolean value <True/False> or <1/0> may be assigned to each of the one or more first time instants. In an embodiment, the value “True” or “1” may correspond to an accurate measurement of the physiological parameter. Whereas, the value “False” or “0” may correspond to an inaccurate measurement of the physiological parameter. Thus, the generated training data may comprise the usage patterns recorded at the one or more first time instants and the Boolean value associated with each of the one or more first time instants. For example, following table illustrates an example record in the training data:

TABLE 1
Example record in the training data

| Usage Pattern | Boolean Value |
|---|---|
| “no_of_mouse_clicks”: 7, && | True |
| “active_on_keyboard”: 0, | |

In certain scenarios, the measure of the physiological parameter may not be determined because of one or more reasons. For example, the user is in motion during the capture of the video by the diagnostic device 104. In another scenario, the user may not present in the video captured by the diagnostic device 104. In such scenarios, the value “False” or “0” may be assigned to the corresponding usage pattern at the first time instant.

Hereinafter, the set of first time instants of the one or more first time instants, that have been assigned the Boolean value of <True/1> have been referred to as opportune time instants.

After generation of the training data, the processor 202 may train the classifier based on the training data. As discussed, the training data comprises the usage pattern at the one or more first time instants and corresponding Boolean value (indicative of successful measurement of the physiological parameter). Therefore, during the training of the classifier, the processor 202 may generate a rule set that may be utilized to determine whether the measurement of the physiological parameter will be successful based on the usage pattern. In an embodiment, the trained classifier may correspond to a decision tree classifier that comprises the rule set. However, a person having ordinary skills in the art will appreciate that the scope of the disclosure is not limited to the classifier being a decision tree classifier. In an embodiment, any other classifier such as a Support Vector Machine (SVM), a Logistic Regression, a Bayesian Classifier, a Copula-based Classifier, a K-Nearest Neighbors (KNN) Classifier, or a Random Forest (RF) Classifier, may be used as the classifier.

In an embodiment, the rule set may define one or more conditions/checks that may be applied to the usage pattern to check whether the measure of the physiological parameter may be determined accurately at the current time instant. Following table illustrates an example rule set:

TABLE 2
An example rule set

| Usage pattern | | |
|---|---|---|
| Keyboard activity | Mouse activity | |
| (KA) | (MA) | Rule set |
| Active = 1; | Active = 1; | IF @timeinstant: T1 |
| Number of key | Number of clicks | KA: Active = 1 && MA: |
| press events | (E2) = 10 | Active = 1 && E1>10 && E2>5 |
| (E1) = 20 | | THEN |
| | | Opportune time Instant = T1 |

Referring to Table 2, the first column and the second column represent the usage pattern. The first column illustrates the keyboard activity (KA) recorded during a pre-defined time frame. The second column illustrates the mouse activity (MA) recorded during the pre-defined time frame. As shown, in the Table 2, the user is active on the keyboard and the number of key press events (E1) is 20. Further, the user is active on the mouse and the number of clicks (E2) is 10. In an embodiment, the classifier may define the rule set as shown in Table 2. In an embodiment, if the user is active on the keyboard and the mouse, and the number of key press events is 10 and the number of mouse clicks is 5 then the time instant T1 may be selected to determine the measure of the physiological parameter.

A person having ordinary skills in the art will appreciate that the scope of the disclosure is not limited to the example rule set illustrated in the Table 2. In an embodiment, the rule set in the Table 2 may include rules related to the other types of operations indicated in the usage pattern. Further, the person having ordinary skills in the art will appreciate that the trained classifier may include more than one rule set, which are generated based on the training data. In an embodiment, the processor 202 may store the trained classifier in the classification unit 210.

After the training of the classifier, in an embodiment, the classification unit 210 may determine whether the physiological parameter of the user can be measured at the current time instant using the trained classifier (i.e., whether the current time instant corresponds to the opportune time instant). In an embodiment, the time instant at which the measure of the physiological parameter may be determined, may be referred to as the opportune time instant. In an embodiment, the classifier may utilize the rule set of determine whether the current time instant corresponds to the opportune time instant.

After determination of the opportune time instant, the classification unit 210 may be configured to transmit a signal to the diagnostic device 104. In an embodiment, the signal may be indicative of an instruction to initiate monitoring of the user at the opportune time instant using the diagnostic device 104 for a pre-defined time duration. Based on the signal transmitted by the classification unit 210 to the diagnostic device 104, the diagnostic device 104 may be configured to capture a video of the user for the pre-defined time duration using the one or more second sensors. For example, an image sensor may be utilized to capture the video. In an embodiment, supplementary information associated with the user may be determined using the one or more second sensors. In an embodiment, the supplementary information may correspond to a biological, physical, and/or chemical signals associated with the user. For example, a temperature sensor may be utilized to capture information associated a body temperature of the user during the pre-defined time period. Such supplementary information may be captured using the one or more second sensors such as a light sensor, an ultrasonic sensor, an audio sensor, and the like. In an embodiment, the video and the supplementary information may be captured by the diagnostic device 104 concurrently using the one or more second sensors.

After capturing the video and the supplementary information by the diagnostic device 104, the diagnostic device 104 may be configured to transmit the video and the supplementary information to the electronic device 102. The physiological parameter monitoring unit 212 may be configured to analyze the video and the supplementary information to determine the set of values associated with the physiological parameter. In an embodiment, the physiological parameter monitoring unit 212 may be configured to utilize one or more known techniques, such as a facial detection technique to determine the set of values. In an embodiment, the facial detection technique may extract facial data from the video which may be utilized to determine the set of values. In an embodiment, the facial data may include one or more facial actions and one or more head gestures. Further, the facial data may include information about hand gestures or body language and body movements of the user. In an embodiment, the physiological parameter monitoring unit 212 may utilize the supplementary information to determine the set of values. For example, a skin temperature may be extracted from the supplementary information. In an embodiment, the skin temperature may correspond to value from the set of values.

The physiological parameter monitoring unit 212 may be configured to determine the measure of the physiological parameter based on the set of values. For example, based on the skin temperature and the analysis of the video, the physiological parameter monitoring unit 212 may be configured to determine a heart rate of the user. A person skilled in the art will understand that the disclosure is not limited to determining the heart rate of the user. In an embodiment, the measure of the physiological parameter may include a respiratory rate, a facial emotion and/or stress associated with the user.

In an alternate embodiment, in order to determine the measure of the physiological parameter, the physiological parameter monitoring unit 212 may be configured to perform face detection of the user by analyzing the video received from the diagnostic device 104. In an embodiment, the physiological parameter monitoring unit 212 may select a particular color channel of the video for analyzing the video. In an embodiment, a plurality of videos that may be overlapping with the captured video within the pre-defined time duration may be processed and a time series signal may be extracted. In an embodiment, a heart rate detection technique may be applied on the extracted time series signal to determine the heart rate of the user. In an embodiment, one or more techniques may be applied on the extracted time series signal to determine the respiratory rate, a facial emotion and/or emotion associated with the user.

After determining the measure of the physiological parameter associated with the user, the physiological parameter monitoring unit 212 may be configured to compare the measure of the physiological parameter with the pre-defined threshold associated with the physiological parameter. Based on the comparison, the physiological parameter monitoring unit 212 may determine whether the measure of the physiological parameter determined is accurate. If the measure of the physiological parameter is inaccurate then the current time instant at which the video was captured may be identified as a false positive. In an embodiment, the physiological parameter monitoring unit 212 may be configured to assign the Boolean value (False or 0) to the usage pattern at the current time instant. In an embodiment, the physiological parameter monitoring unit 212 may be configured to assign the Boolean value (True or 1) to the usage pattern of the current time instant if the measure of the physiological parameter is accurate.

In an embodiment, the physiological parameter monitoring unit 212 may generate a feedback that includes the information pertaining to the usage pattern at the current time instant and the Boolean value assigned to the usage pattern at the current time instant. In an embodiment, the physiological parameter monitoring unit 212 may update/train the classifier based on the feedback. In an embodiment, during updating of the classifier, a new rule set may be defined to determine the opportune time instants for the subsequent time instants accurately.

In an embodiment, if the determined measure of the physiological parameter is accurate, then the measure of the physiological parameter may be displayed on the display screen of the electronic device 102. Additionally, a trend of the measure of the physiological parameter till the current time instant may be displayed to the user by means of one or more graphs, such as a bar graph, a line graph, and the like.

A person skilled in the art will understand that the scope of the disclosure should not be limited to determining the measure of the physiological parameter based on the aforementioned factors and using the aforementioned techniques. Further, the examples provided in supra are for illustrative purposes and should not be construed to limit the scope of the disclosure.

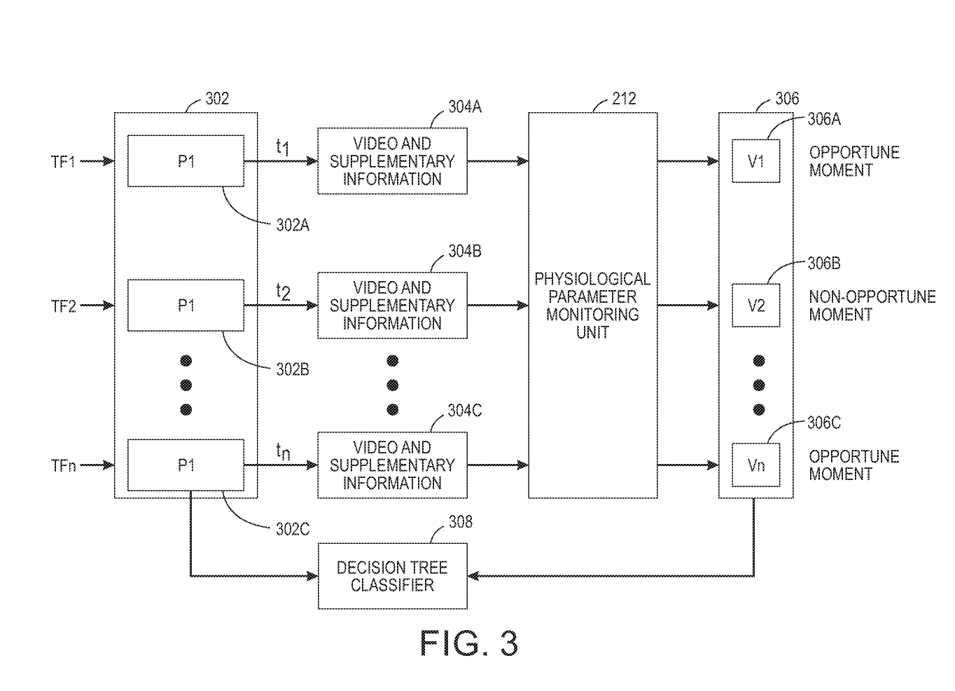

FIG. 3 illustrates a block diagram to train the classifier, in accordance with at least one embodiment. FIG. 3 is described in conjunction with FIG. 1 and FIG. 2.

With reference to FIG. 3, at time frames TF1, TF2, . . . , TFn the usage pattern P1 (denoted by 302a), usage pattern P2, (denoted by 302b) . . . , usage pattern Pn (denoted by 302n) may be determined by the activity monitoring unit 208, respectively. Further, the activity monitoring unit 208 may be configured to extract the one or more time instants t1, t2, . . . , tn corresponding to each of the usage patterns. In an embodiment at each of the one or more time instants t1, t2, . . . , tn, the user may be monitored using the diagnostic device 104. Based on the monitoring, for each time instant, a video and supplementary information may be captured by the diagnostic device 104. For example, 304a, 304b . . . 304n may correspond to the video and the supplementary information of the one or more time instants t1, t2, . . . , tn, respectively.

Further based on the analysis of each of the video and the supplementary information by the physiological parameter monitoring unit 212, the physiological parameter monitoring unit 212 may be configured to determine the measure of the physiological parameter V1, V2, . . . , Vn at the one or more time instants t1, t2, . . . , tn respectively. Further, the physiological parameter monitoring unit 212 may determine the accuracy of each of the measure of the physiological parameter V1, V2, . . . , Vn. If the measure of the physiological parameter determined by the physiological parameter monitoring unit 212 is accurate then the time instant corresponding to the measure of the physiological parameter may be categorized as the opportune time instant. For example, with reference to FIG. 3, the measure of the physiological parameter V1 is determined accurately and thus the time instant t1 may be categorized as the opportune time instant. Similarly, the measure of the physiological parameter V2 is determined inaccurately and thus the time instant t2 may be categorized as the non-opportune time instant. Further, the measure of the physiological parameter Vn is determined accurately and thus the time instant to may be categorized as the opportune time instant.

Thus, based on the categorization of the time instants into the opportune time instant and the non-opportune time instant, and the usage pattern to generate the decision tree classifier 308. As discussed above in conjunction with FIG. 2, the decision tree classifier 308 may comprise the rule set. Based on the generated decision tree classifier 308, the electronic device 102 may be configured to determine the opportune time instant for the real-time data (e.g., the current time instant).

A person skilled in the art will understand that the scope of the disclosure should not be limited to utilizing the disclosed method to train the classifier. Further, the examples provided in supra are for illustrative purposes and should not be construed to limit the scope of the disclosure.

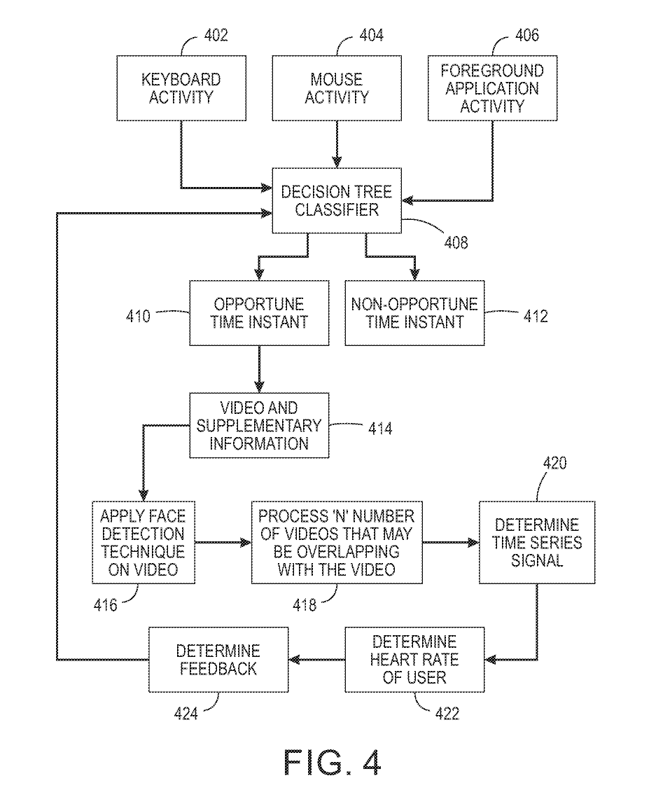

FIG. 4 is a block diagram that illustrates an exemplary scenario to monitor the heart rate of the user, in accordance with at least one embodiment. The FIG. 4 is described in conjunction with FIG. 1 and FIG. 2.

With reference to FIG. 4, the activity monitoring unit 208 may be configured to monitor a keyboard activity 402, a mouse activity 404, and a foreground application activity 406 associated with the user using the one or more first sensors, such as a pressure sensor. The activity monitoring unit 208 may sample the activity of the one or more input devices and the foreground applications into the pre-defined time duration.

In an embodiment, the keyboard activity 402 that may be monitored by the pressure sensor may be represented in the form of a JSON object as below:

{
Number of keys pressed = 56
Active on keyboard = 1
Timestamp: 201-02-03; 16:35:45
}

The JSON object as shown above captures the keyboard activity 402 such as the number of keys pressed, whether the user is active on the keyboard, and a timestamp associated with the keyboard activity 402.

A person skilled in the art will understand that the scope of the disclosure should not be limited to determining the keyboard activity 402 using the aforementioned techniques. Further, the examples provided in supra are for illustrative purposes and should not be construed to limit the scope of the disclosure.

In an embodiment, the mouse activity 404 that may be monitored by the pressure sensor may be represented in the form of a JSON object as below:

{
Number of left clicks = 10
Number of right clicks = 3
Number of scroll events = 5
Active on mouse = 1
Timestamp: 201-02-03; 16:35:45
}

The JSON object as shown above captures the mouse activity 404 such as a number of left clicks, a number of right clicks, a number of scroll events, whether the user is active on the mouse, and a timestamp associated with the mouse activity 404.

A person skilled in the art will understand that the scope of the disclosure should not be limited to determining the mouse activity 404 using the aforementioned techniques. Further, the examples provided in supra are for illustrative purposes and should not be construed to limit the scope of the disclosure.

In an embodiment, the foreground application activity 406 that may be monitored by the electronic device 102 may be represented in the form of a JSON object as below:

{
Open application title = “Google Chrome”, website name.
Open application title = “Inbox”, email id.
Timestamp: 201-02-03; 16:35:45
}

The JSON object as shown above captures the foreground application activity 406 such as a title of the foreground application that is being accessed, and a timestamp associated with the foreground application activity 406.

A person skilled in the art will understand that the scope of the disclosure should not be limited to determining the foreground application activity 406 using the aforementioned techniques. Further, the examples provided in supra are for illustrative purposes and should not be construed to limit the scope of the disclosure.

Based on the keyboard activity 402, the mouse activity 404, and the foreground application activity 406, the activity monitoring unit 208 may be configured to generate the usage pattern. Based on the usage pattern the activity monitoring unit 208 may be configured to determine the current time instant. The usage pattern is given as an input to the decision tree classifier 408. The decision tree classifier 408 may categorize the current time instant as the opportune time instant 410 or the non-opportune time instant 412. In an embodiment, the opportune time instant 410 may correspond to the time instant at which the user may be monitored using the diagnostic device 104. The time instants that may be categorized as the non-opportune time instant 412 may not be utilized for monitoring the user.

After determining the current time instants as the opportune time instant, the electronic device 102 may transmit the signal to monitor the user using the diagnostic device 104 at the current time instant. In response to the signal, the diagnostic device 104 may capture the video and the supplementary information 414 of the user for the pre-defined duration. In an embodiment, a plurality of videos and the supplementary information captured by the diagnostic device 104 for the pre-defined duration (e.g., 15 seconds) may be analyzed by the processor 202 and a face detection technique 416 may be applied on the plurality of videos.

Further at block 418, the processor 202 may be configured to process ‘n’ number of videos that may be overlapping with the video and the supplementary information 414 within the pre-defined time duration. In an embodiment, at block 420, the processor 202 may be configured to determine the time series signal based on the processing of the ‘n’ number of videos. At block 422, the physiological parameter monitoring unit 212 may be configured to implement one or more heart rate detection techniques on the captured video and the supplementary information 414. Based on the implementation of the heart rate detection technique, the physiological parameter monitoring unit 212 may be configured to determine the heart rate of the user. For example, the heart rate detection technique may determine a skin temperature of the user by analyzing the video and the heart rate may be determined based on the implementation of the heart rate detection technique on the skin temperature.

In an embodiment, at block 424, the determined heart rate may be compared with a pre-defined threshold to check the accuracy of the determined heart rate. In an embodiment, if the determined heart rate is inaccurate then the feedback may be transmitted to the decision tree classifier. Based on the feedback, the processor 202 may train/update the decision tree classifier.

A person skilled in the art will understand that the scope of the disclosure should not be limited to determining the heart rate of the user using the aforementioned techniques. Further, the examples provided in supra are for illustrative purposes and should not be construed to limit the scope of the disclosure.

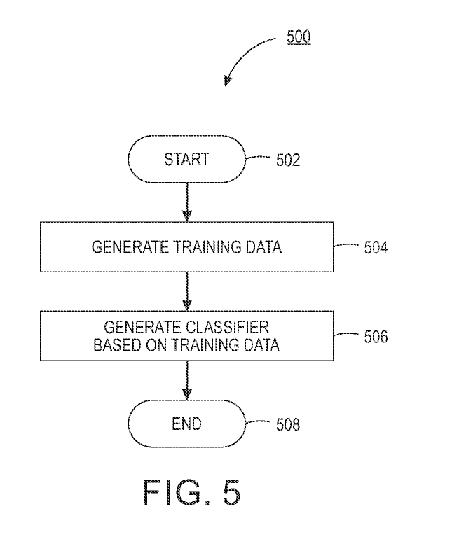

FIG. 5 is a flowchart 500 that illustrates a method to generate the classifier in accordance with at least one embodiment. The flowchart 500 is described in conjunction with FIG. 1 and FIG. 2.

The method starts at step 502 and proceeds to step 504. At step 504, the electronic device 102 may generate training data. The training data may be generated based on the usage pattern determined at the one or more first time instants. Based on the usage pattern, the video and supplementary information may be determined at the one or more first time instants and the opportune time instant may be determined. At step 506, the processor 202 may be configured to generate the classifier based on the training data. Control passes to end step 508.

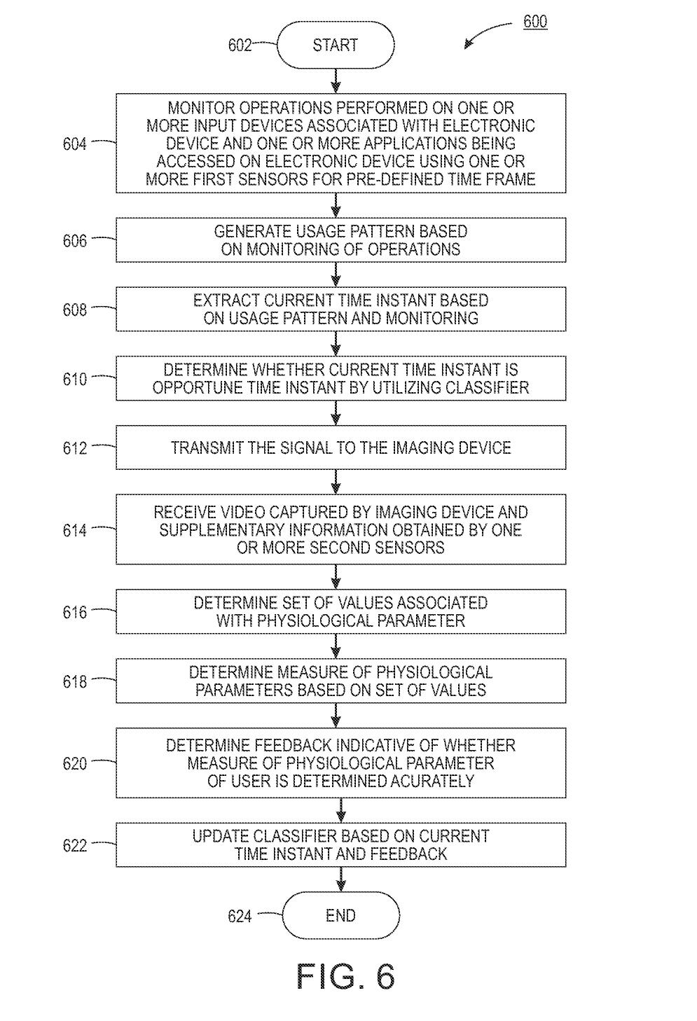

FIG. 6 is a flowchart 600 that illustrates a method to monitor one or more physiological parameters in accordance with at least one embodiment. The flowchart 500 is described in conjunction with FIG. 1 and FIG. 2. The method starts at step 602 and proceeds to step 604.

At step 604, the electronic device 102 may be configured to monitor the operations performed on the one or more input devices associated with the electronic device 102 and the one or more applications being accessed on the electronic device 102, by the user, using the one or more first sensors for a pre-defined time frame. At step 606, the electronic device 102 may be configured to generate the usage pattern based on the monitoring of the operations and the one or more applications being accessed on the electronic device 102. At step 608, the electronic device 102 may be configured to extract the current time instant based on the usage pattern and the monitoring. At step 610, the electronic device 102 may be configured to determine whether the current time instant is the opportune time instant by utilizing the classifier.

At step 612, the electronic device 102 may be configured to transmit the signal to the diagnostic device 104. In an embodiment, the signal may be indicative of initiating monitoring the user at the current time instant using the one or more second sensors for a pre-defined time duration. At step 614, the electronic device 102 may be configured to receive the video captured by the diagnostic device 104 and the supplementary information obtained by the one or more second sensors. At step 616, the electronic device 102 may be configured to analyze the video and the supplementary information to determine the set of values associated with the physiological parameter.

At step 618, the electronic device 102 may be configured to determine the measure of physiological parameter of the user based on the set of values. At step 620, the electronic device 102 may be configured to determine feedback that may be indicative of whether the measure of physiological parameter is determined accurately. At step 622, the electronic device 102 may be configured to update/train the classifier based on the current time instant and the feedback. Control passes to end step 624.

Various embodiments of the disclosure provide a non-transitory computer readable medium and/or storage medium, and/or a non-transitory machine-readable medium and/or storage medium having stored thereon, a machine code and/or a computer program having at least one code section executable by a machine and/or a computer to monitor a physiological parameter of a user. The at least one code section in an electronic device 102 causes the machine and/or computer comprising one or more processors to perform the steps, which comprises monitoring, by one or more first sensors, an operation performed on one or more input devices associated with an electronic device 102 and one or more applications being accessed on the electronic device 102 for a pre-defined time frame. The one or more processors may identify a current time instant based on the monitoring of the operation. Further the one or more processors may determine whether the current time instant corresponds to an opportune time instant by utilizing a classifier. In an embodiment, the physiological parameter of the user may be monitored at the current time instant when the current time instant corresponds to the opportune time instant.

Various embodiments of the disclosure encompass numerous advantages including methods and systems for monitoring the physiological parameter of the user. In the disclosed embodiments, the opportune time instant may be determined and the user may be monitored at the opportune time instant. Further, the user is unaware of the monitoring but the user gets information pertaining to the measure of the physiological parameter. Additionally, as the measure of the physiological parameter is determined only at the opportune time instant, the amount of digital storage required for maintaining the information pertaining to the measure of the physiological parameter is minimized.

The present disclosure may be realized in hardware, or in a combination of hardware and software. The present disclosure may be realized in a centralized fashion, in at least one computer system, or in a distributed fashion, where different elements may be spread across several interconnected computer systems. A computer system or other apparatus adapted for carrying out the methods described herein may be suited. A combination of hardware and software may be a general-purpose computer system with a computer program that, when loaded and executed, may control the computer system such that it carries out the methods described herein. The present disclosure may be realized in hardware that comprises a portion of an integrated circuit that also performs other functions.

A person with ordinary skill in the art will appreciate that the systems, modules, and sub-modules have been illustrated and explained to serve as examples and should not be considered limiting in any manner. It will be further appreciated that the variants of the above disclosed system elements, modules, and other features and functions, or alternatives thereof, may be combined to create other different systems or applications.

Those skilled in the art will appreciate that any of the aforementioned steps and/or system modules may be suitably replaced, reordered, or removed, and additional steps and/or system modules may be inserted, depending on the needs of a particular application. In addition, the systems of the aforementioned embodiments may be implemented using a wide variety of suitable processes and system modules, and are not limited to any particular computer hardware, software, middleware, firmware, microcode, and the like. The claims can encompass embodiments for hardware and software, or a combination thereof.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. A method for monitoring a physiological parameter of a user, the method comprising:
monitoring, by one or more first sensors, an operation performed on one or more input devices associated with an electronic device and one or more applications being accessed on the electronic device for a pre-defined time frame;
identifying, by one or more processors, a current time instant based on the monitoring of the operation and the one or more applications being accessed; and
determining, by the one or more processors, whether the current time instant corresponds to an opportune time instant by utilizing a classifier, wherein the physiological parameter of the user is monitored at the current time instant when the current time instant corresponds to the opportune time instant.

2. The method of claim 1, further comprising generating, by the one or more processors, a usage pattern of the electronic device based on the monitoring of the operation and the one or more applications being accessed.

3. The method of claim 1, further comprising monitoring, by one or more second sensors, one or more features of the user at the determined opportune time instant for a pre-defined duration to obtain a set of values, wherein the one or more features associated with the user comprise, a skin tone, facial features, and a measure of temperature.

4. The method of claim 3, wherein the one or more second sensors comprise an image sensor, a temperature sensor, a light sensor, an audio sensor and/or an ultrasonic sensor.

5. The method of claim 3, further comprising determining, by the one or more processors, a measure of the physiological parameter of the user based on the set of values.

6. The method of claim 5, further comprising determining, by the one or more processors, a feedback indicative of whether the measure of the physiological parameter of the user is determined successfully.

7. The method of claim 6, further comprising updating, by the one or more processors, the classifier based on the determined opportune time instant, and the feedback.

8. The method of claim 1, wherein the operation corresponds to a click event, a scroll event, a typing event, and a touch event.

9. The method of claim 1, wherein the one or more input devices comprise a keyboard, a mouse, a track pad, a track ball, and/or a touch display.

10. The method of claim 9, wherein the one or more first sensors monitor a keyboard activity of the user, a mouse activity of the user, a touch pad activity of the user, a track ball activity of the user, a presence of the user, a motion of the user, and an ambience.

11. The method of claim 1, wherein the one or more first sensors comprise a pressure sensor, and a motion sensor.

12. The method of claim 1, wherein the physiological parameter comprises a heart rate, a respiratory rate, a facial emotion and/or stress.

13. An electronic device to monitor a physiological parameter of a user, the electronic device comprising:
one or more processors configured to:
monitor an operation performed on one or more input devices associated with the electronic device and one or more applications being accessed on the electronic device for a pre-defined time frame;
identify a current time instant based on the monitoring of the operation and the one or more applications being accessed; and
determine whether the current time instant corresponds to an opportune time instant by utilizing a classifier, wherein the physiological parameter of the user is monitored at the current time instant when the current time instant corresponds to the opportune time instant.

14. The electronic device of claim 13, wherein the one or more processors are configured to generate a usage pattern of the electronic device based on the monitoring.

15. The electronic device of claim 13, wherein the one or more processors are configured to monitor the user utilizing one or more second sensors at the determined opportune time instant for a pre-defined duration to obtain a set of values.

16. The electronic device of claim 15, wherein the one or more processors are configured to determine a measure of the physiological parameter of the user based on the set of values.

17. The electronic device of claim 16, wherein the one or more processors are configured to determine a feedback indicative of whether the measure of the physiological parameter of the user is monitored successfully.

18. The electronic device of claim 17, wherein the one or more processors are configured to update the classifier based on the determined opportune time instant, and the feedback.

19. The electronic device of claim 13, wherein the one or more input devices comprise a keyboard, a mouse, a track pad, a track ball, and/or a touch display.

20. The electronic device of claim 19, wherein the one or more first sensors monitor a keyboard activity of the user, a mouse activity of the user, a touch pad activity of the user, a track ball activity of the user, a presence of the user, a motion of the user, and an ambience.

21. A non-transitory computer-readable storage medium having stored thereon, a set of computer-executable instructions for causing a computer comprising one or more processors to perform steps comprising:
monitoring, by one or more first sensors, an operation performed on one or more input devices associated with an electronic device and one or more applications being accessed on the electronic device for a pre-defined time frame;
identifying, by one or more processors, a current time instant based on the monitoring of the operation and the one or more applications being accessed; and
determining, by the one or more processors, whether the current time instant corresponds to an opportune time instant by utilizing a classifier, wherein a physiological parameter of a user is monitored at the current time instant when the current time instant corresponds to the opportune time instant.
